(19) United States
(12) Patent Application Publication
(10) Pub. No.: US 2003/0236500 A1
(43) Pub. Date: Dec. 25, 2003

(54) Injection device

(76) Inventor: Rolf Rainer Scheu, Frankfurt (DE)

Correspondence Address: Pauley Petersen Kinne & Erickson, 2800 W. Higgins Road, Suite 365, Hoffman Estates, IL, 60195, US

(21) Appl. No.: 10/429,928

(22) Filed: May 5, 2003

(30) Foreign Application Priority Data
May 6, 2002 (DE) 202 07 083.2
May 24, 2002 (DE) 202 08 105.2

(51) Int. Cl. A61M 5/00

(52) U.S. Cl. 604/190; 604/177

(57) ABSTRACT

An injection device for injecting an injection solution prepared from a powdered medication in sterile water immediately before use which is administered by a syringe. A winged cannula has a hollow needle and a needle holder with wings. A female Luer Lock connector is joined to the winged cannula by a flexible connecting tube and has an insertion region for the connecting tube and a connection region for placing the syringe, filled with the injection solution, against a skin surface, and also has a continuous bore that is continuous from the insertion region to the connection region of the Luer Lock connector. A porous filter element permeable to the injection solution is provided in the continuous bore of the Luer Lock connector.

### BACKGROUND OF THE INVENTION

1. Field of the Invention

This invention relates to an injection device for injecting injection solutions, which are prepared from powdered medications in sterile water immediately before use and are administered with a syringe, including a winged cannula with a hollow needle and a needle holder with wings and including a female Luer Lock connector, joined to the winged cannula by a flexible connecting tube and having an insertion region for the connecting tube and a connection region for placing the syringe, filled with injection solution, against the skin, and having a continuous bore that is continuous from the insertion region to the connection region of the Luer Lock connector.

2. Discussion of Related Art

By way of example, injection devices of this generic type are needed for administering coagulants, which are injected into a patient's blood vessel. Coagulants come in powder form, and only immediately before use are they dissolved in water so as then to be administered, because in dissolved form these medications do not have a long storage life.

For injecting injection solutions that are put into solution using sterile water only immediately before use, some protection against contamination is necessary. Before administering to the patient, the freshly prepared injection solution must be subjected to a cleaning process to remove possible foreign bodies, such as solid contaminant particles.

One object of this invention is to provide administration by injection of injection solutions prepared from powdered medications immediately before use, using an easily manipulated injection device, in which the injection solution is cleaned or kept clean simultaneously. Because such injection devices are disposable items, it should also be possible to construct and produce the injection devices simply, economically, and inexpensively.

The use of filters for medical applications for the sake of decontamination is known. For instance, German Patent Disclosure DE 24 01 782 A discloses a filter device for medical infusion and injection devices with a needle holder with a hollow needle and a syringe is known, in which a cylindrical, cup-shaped, porous filter of sintered metal powder is provided. The filter is inserted on one side into the needle holder, provided with a continuous bore, and the hollow needle is secured on the other side in the bore of the needle holder. The syringe is then introduced into the bore, equipped with the filter, of the needle holder, and the filter is intended to filter out particulate contaminants from the parenteral liquids delivered to the patient. From U.S. Pat. No. 4,435,176, an injection device including a hollow needle and a needle holder and a syringe analogous to that taught by German Patent Disclosure DE 24 01 782 A is also known, but in it a platelike filter is used. These known injection devices form a compact unit, and the syringe is connected directly to the hollow needle via the needle holder and is thus immediately adjacent the point of injection into the patient, making handling more difficult.

From German Utility Model DE 297 201 82 U1, a further injection device with a syringe and a needle holder with a hollow needle is known. Between the needle holder and the syringe, a filter embodied as a separate part is inserted. In this case, the hollow needle, needle holder, filter and syringe form a compact, dimensionally stable unit, and the syringe is also close to the patient. The compact, dimensionally stable unit is difficult for a patient to handle, alone.

U.S. Pat. Nos. 5,603,792 and 5,500,003 disclose a protector in the form of a diaphragm for a pressure pickup for measuring the blood pressure of a patient in blood dialysis procedures. The diaphragm simultaneously takes on the function of a sterile barrier, which protects both the patient and the dialysis equipment against the danger of contamination with virus-infected blood. The diaphragm is welded in place between two polycarbonate flanges that are permanently mounted at the outlet toward the patient of a female Luer Lock connector, and to the other outlet the dialysis can be connected for pressure measurement.

### SUMMARY OF THE INVENTION

One object of this invention is attained by refining a generic injection device according to the characteristics of this invention as described in the claims and in this specification.

According to this invention, for cleaning the injection solution to be injected, there is a porous filter element that is permeable to the injection solution in the continuous bore of the Luer Lock connector. The syringe can then be attached directly to the female Luer Lock connector in the connection region, and the injection solution expressed from the syringe is forced through the porous filter element and passes from the Luer Lock connector via the connecting tube into the hollow needle, equipped with the winged cannula, to reach the patient. The embodiment of the filter element with a permeability that does not hinder the flow of the injection solution yet at the same time traps any solid particles still contained in the injection and prevents them from passing through the filter element, is important to this invention. According to this invention, the filter element must be equipped with adequate permeability and must have a pore size that must not be below a certain minimum, so as not to hinder the flow of the injection solution, yet also must not be exceeded, either, in order to catch the solid particles. Preferably, the filter element has pores with a mean diameter of 2 to 100 μm, and preferably pores with a mean diameter of 5 to 20 μm.

The injection device equipped according to this invention enables easy manipulation by the patient, alone, for injecting injection solutions, because with the wings of the winged cannula, guidance of the needle and puncturing of the intended blood vessel can be simply accomplished. The female Luer Lock connector, joined flexibly to the winged cannula by the connecting tube, permits a syringe to be connected to a Luer Lock connector or Luer connector that contains the medication to be injected in the form of an injection solution. The filter element disposed in the Luer Lock connector reliably traps solids that, for example, during preparation of the injection solution remain behind in the syringe to be attached. This is often the case, for example, in preparing coagulants for hemophiliac patients, because the medication is initially in powdered form and is prepared with water to make an injectable solution, but solids can still remain in this solution and are reliably trapped by the filter element.

The injection device of this invention enables particularly easy manipulation and administration of a medication, which can optionally be performed by the patient himself.

In particular, the injection device of this invention makes it possible use medications that are in powdered form before their administration and that are prepared into an injection solution by adding water only immediately before use.

For this purpose, by way of example, a disposable glass syringe that is prefilled with the powdered medication can be used. For administering the medication, sterile water is first drawn up into the syringe, and the contents are then thoroughly shaken, in order to obtain an injection solution comprising medication and water. Then the syringe is joined to the connection region of the Luer Lock connector of the injection device of this invention, and the injection solution can be administered via the hollow needle of the winged cannula, the hollow needle being inserted into a blood vessel of the patient. Any solid particles that remain in the injection solution are reliably trapped by the filter element that is built into the Luer Lock connector.

Because of the winged cannula, the injection device of this invention is easily manipulated and guided, and easy decoupling of the winged cannula from the syringe connected to the Luer Lock connector is also made possible by the flexible connecting tube. One essential characteristic of this invention is the disposition of the filter element inside the Luer Lock connector. Preferably, the filter element is disposed at the transition from the insertion region of the Luer Lock connector to the connection region of the Luer Lock connector for the syringe, and the continuous bore of the Luer Lock connector widens in the direction of the connection region and forms a steplike shoulder. The filter element can contact this shoulder. The filter element is preferably embodied in a plate form.

The filter element is durably fixed in its position inside the Luer Lock connector. For example, the filter element is embedded, along its outer circumference, in the Luer Lock connector. This can be done, for example, so that the Luer Lock connector is made as an injection-molded part of a thermoplastic, and the filter element is placed in the mold in the production of the injection-molded part and partially spray-coated, and is partially embedded, especially along its outer circumference, in the thermoplastic of the Luer Lock connector. The region of the filter element that covers the continuous bore of the Luer Lock connector remains free and thus remains permeable to the injection solution to be injected.

The filter element, which is disposed inside the Luer Lock connector, is preferably embodied from a woven fabric on the basis of polyamide, such as nylon, and the pore size, or open mesh area, of the filter element is selected to be in the range from 2 to 100 μm. Particles with a larger area than the open mesh size of the fabric comprising the filter element are trapped by the filter element.

It is also possible for a filter element in the form of a disk made from sintered plastic granulate or metal powder to be disposed inside the connector. The selected pore size of the filter element assures that the injection solution can still be applied from the syringe attached to the Luer Lock connector without problems, using a patient's usual thumb pressure, and at the same time the critical solid particles to be expected are reliably trapped by the filter element.

Also, membranes of plastic film can be used as a filter element, as long as they are equipped with pores in sufficient number and a sufficient size of 2 to 100 μm, so as to allow the injection solution to pass through the filter element.

The connecting tube provided for the injection device of this invention is produced from a flexible plastic, such as soft PVC or a thermoplastic/elastic polyurethane and has a length of 30 to 400 mm, preferably 300 to 400 mm. The outer diameter of the connecting tube is in the range of 1.0 to 2.5 mm, for an inner diameter of 0.2 to 1.5 mm. The dimensions of the connecting tube are selected to obtain the smallest possible inside volume that would act as an idle space for the injection solution to be injected, while at the same time it is possible to perform the injection through the connecting tube with still reasonable expenditure of force. The connecting tube is introduced into the bore of the insertion region of the Luer Lock connector and durably secured there, for instance by high-frequency or ultrasonic welding, depending on the material used for the connecting tube and the Luer Lock connector. It is also possible to equip the Luer Lock connector, on the outside of its insertion region, with two formed-on wings located in the same plane.

The Luer Lock connector for the injection device of this invention is preferably produced by injection molding from a thermoplastic, such as acrylate-butadiene-styrene, polycarbonate, or polymethacrylate. The filter element disposed in the interior of the Luer Lock connector can preferably be integrally embedded and partly spray-coated in the process of producing the connector. According to one embodiment of this invention, the female Luer Lock connector with the filter element is formed of a first injection-molded part of a thermoplastic, forming the insertion region, in which the filter element placed on its inlet side is embedded by spraying on a second injection-molded part, forming the connection region, including the same thermoplastic as the insertion region, or a different one, and the two injection-molded parts are united to form the connector.

Still other ways of introducing the filter element into the bore of the Luer Lock connector are also possible. For example, the Luer Lock connector can be produced as an injection-molded part from a thermoplastic, and the filter element can be subsequently inserted into the continuous bore of the Luer Lock connector and durably secured therein, for instance by high-frequency welding.

The hollow needle provided at the winged cannula of the injection device of this invention can have an active length, with which it protrudes beyond the needle holder, of 15 to 20 mm, for an outer diameter of 0.4 to 1.2 mm, preferably 0.5 to 0.6 mm, and the inner diameter can amount to 0.28 to 0.38 mm.

The hollow needle of the injection device is protected in its active region, protruding past the needle holder, before use by a removable protective sheath. It is also possible for the hollow needle and in particular its tip also to have a protective cover after the injection device has been used. This protective cover can, for example, include a cover element or flap protector disposed on the needle holder. It is also possible to embody the winged cannula so that the hollow needle is retractable into the needle holder after use, or the needle holder is equipped with a covering part that can be advanced over the hollow needle.

### BRIEF DESCRIPTION OF THE DRAWINGS

Further details of the injection device of this invention are described below in view of exemplary embodiments shown in the drawings, wherein:

FIG. 1 shows a diagrammatic view of the injection device of this invention;

FIG. 2 shows an enlarged view of the needle holder shown in FIG. 1;

FIG. 3 shows an enlarged view of the winged cannula shown in FIG. 1;

FIG. 4 shows a female Luer Lock connector and filter element shown in FIG. 1, in an enlarged sectional view;

FIG. 5 shows a sectional view of one embodiment of the Luer Lock connector shown in FIG. 4;

FIG. 6 shows a female Luer Lock connector with-wings for the injection device shown in FIG. 1; and

FIG. 7 shows a schematic view of the injection device of this invention, including the syringe, in use.

### DESCRIPTION OF PREFERRED EMBODIMENTS

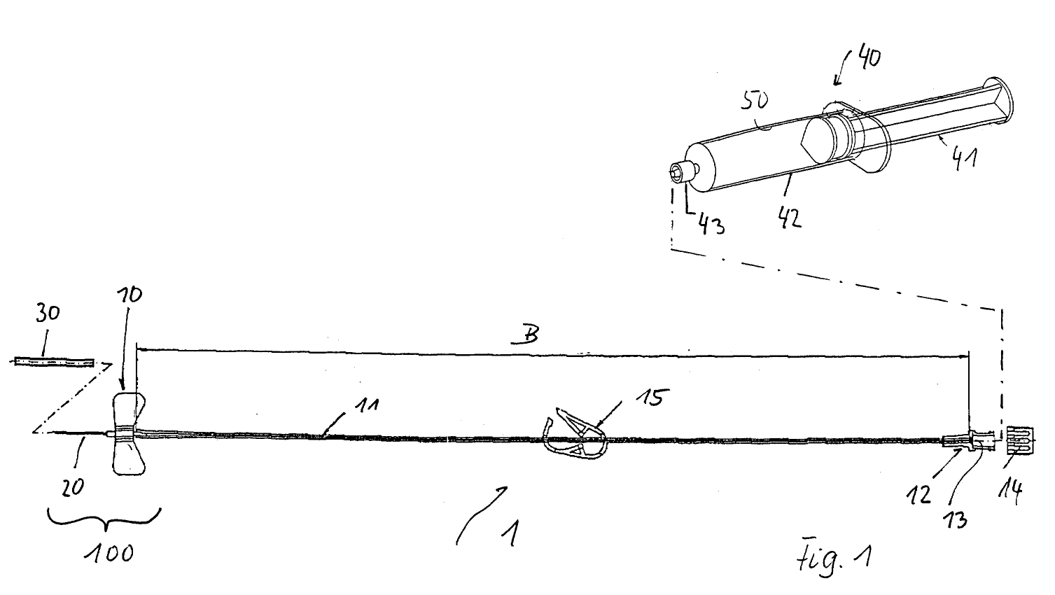

The injection device shown in FIG. 1 includes a winged cannula 100 and a female Luer Lock connector 12, which are joined by a flexible connecting tube 11, forming a lumen that is open from the connector 12 via the connecting tube 11 as far as the tip 20a of the winged cannula 100. The injection device 1 of FIG. 1 is a disposable item. The winged cannula 100 comprises the needle holder 10, with formed-on wings 101a, 101b that are located in the same plane, and the hollow needle 20 inserted into the needle holder 10. The hollow needle 20, in its active portion protruding from the needle holder 10, is covered before use by a tubular protective sheath 30.

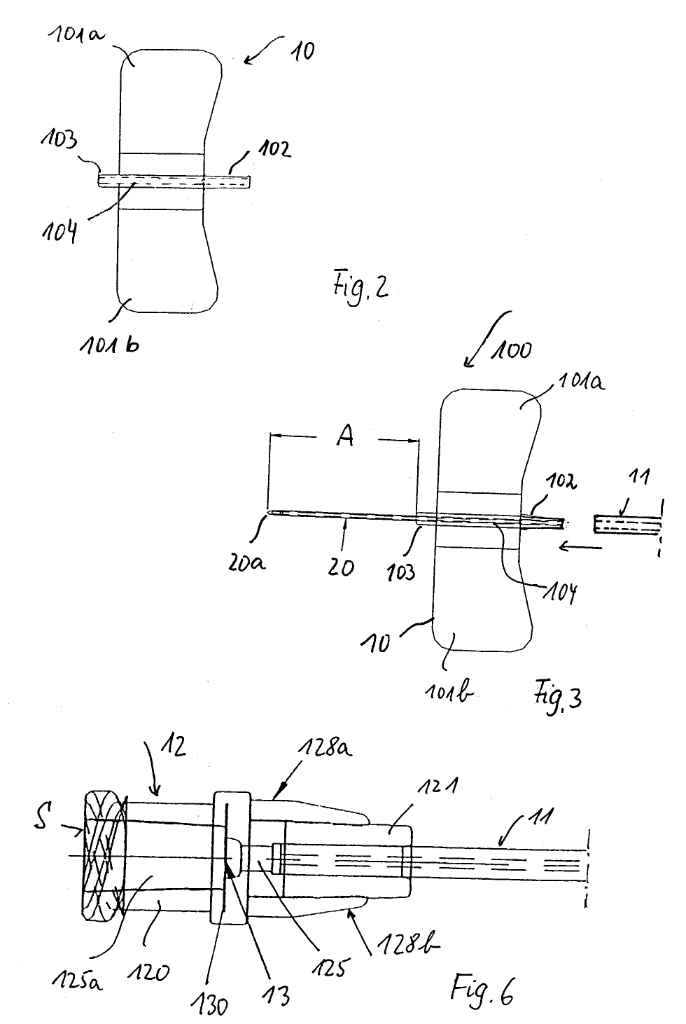

As shown in FIG. 2, the needle holder 10 has an axially continuous bore 104, to both sides of which the wings 101a and 101b extend. The bore 104 protrudes on both sides past the wings 101a and 101b and the needle holder 10, each with a respective connection stub 102 and 103. The hollow needle 20 is introduced into the connection stub 103, as FIG. 3 shows, and is thrust through the bore 104, preferably as far as the outlet of the connection stub 102, so that the needle 20 is solidly joined to the needle holder 10. The needle 20 can also be installed together with the needle holder 10 in the injection molding process and spray-coated. The active length A of the hollow needle 20 is approximately 15 to 30 mm, preferably approximately 18 to 20 mm, for an outer diameter of 0.5 mm and an inner diameter of 0.28 mm. The needle holder 10 and the fixedly inserted hollow needle 20 form the so-called winged cannula 100. On the side of the needle holder 10 remote from the needle, the connecting tube 11 with the continuous lumen is slipped onto the connection stub 102 and joined to it in a firmly adhering manner, for instance by high-frequency welding. The needle holder 10 is also produced from a thermoplastic in a dimensionally stable embodiment, for example from soft PVC.

The connecting tube has a preferred length B in the range from 30 to 400 mm, for instance 350 mm. As shown in FIG. 1, an on-off clamp 15 for clamping off the connecting tube can be mounted on the connecting tube 11.

On the end of the connecting tube 11 remote from the winged cannula 100, as FIG. 1 shows, a female Luer Lock connector 12 with a continuous bore is provided, with which the end of the connecting tube 11 is solidly joined. The Luer Lock connector 12 has a filter element 13, and when not in use can be covered with a protective cap 14 on its free end. When the device is put to use, the protective cap 14 is removed, and in its place, the syringe 40, filled with an injection solution 50, is connected along with its tip 43, embodied for example as a Luer lock, to the connector 12.

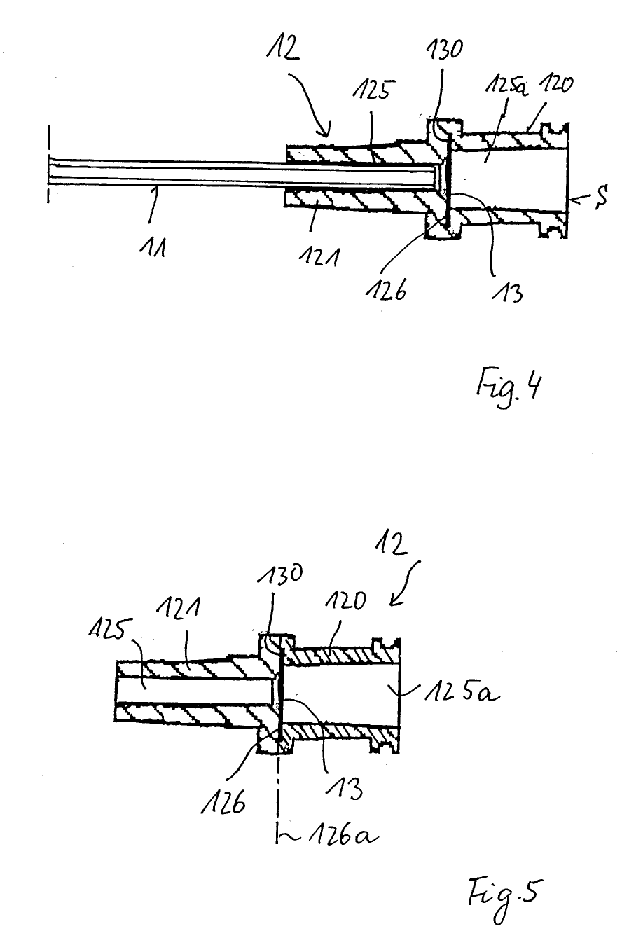

In FIG. 4, the connector 12 with the inserted connecting tube 11 of FIG. 1 is shown enlarged. The female Luer Lock connector 12 has an insertion region 121 for receiving the connecting tube 11 and, adjoining the insertion region 121, a connection region 120 for placing the syringe 40 against the skin. The connector 12 has a bore 125, which is axially continuous from the insertion region 121 to the connection region 120 and which widens at the transition from the insertion region 121 to the connection region 121, widened bore 125a, forming a steplike shoulder 126. The filter element 13 is accommodated inside the connector 12 in the bore 125, preferably in the transition region between the insertion region 121 and connection region 120, and the filter element 13 contacts the steplike shoulder 126. The filter element 13 is intended to be durably joined to the connector 12 and nondisplaceable. The filter element 13, which is embodied in the form of a disk, for instance from a woven fabric of polyamide, such as nylon, with a pore size of 20 μm, has a larger diameter than the widened bore 125a of the connection region 120. The region 130 of the outer circumference of the filter element 13 that protrudes beyond the bore 125a is embedded in the connector 12, such as in the wall of the connector 12. The middle region of the filter element 13 spans the continuous bore 125, 125a of the connector 12 and enables the flow of the injection solution, introduced by a syringe, through into the connecting tube 11. During this flow through the filter element 13, unwanted particles are filtered out of the injection solution and trapped in the filter element 13, so that they cannot get into a circulatory system of the patient. The syringe end of the connector 12 is marked S. The connector 12 is produced as an injection-molded part from a thermoplastic, and preferably from dimensionally stable rigid plastics. To make the embedding and fixing of the filter element 13 in the connector possible, the connector 12 is for example, as shown in FIG. 5, constructed in production from multiple parts which are joined together. For example, first the insertion region 121 is made as an injection-molded part of a thermoplastic such as polycarbonate, and next, the disklike filter element is placed on the inlet side 126a of the insertion region, namely the side toward the syringe 40. Then, the insertion region 120 is cast as a second injection-molded part, preferably from the same material as the insertion region 121, and in the process the filter element 13 is embedded along its circumference 130 between the two injection-molded parts 121 and 120.

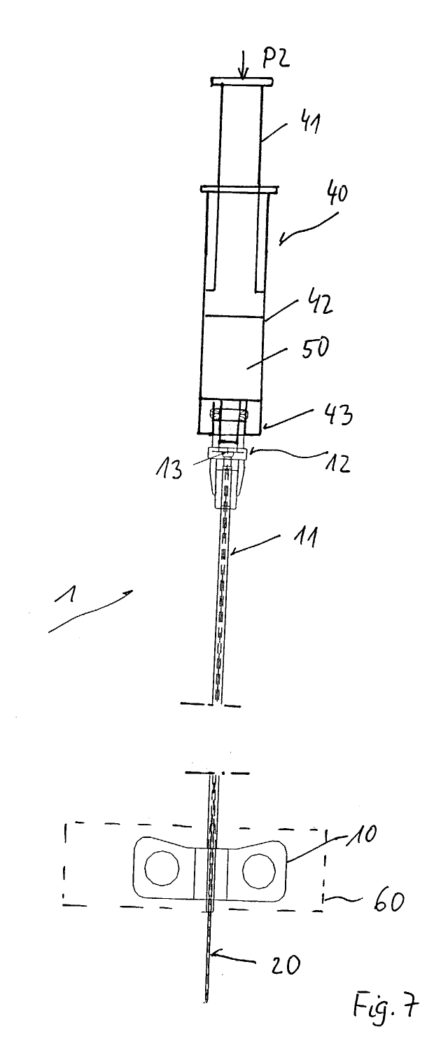

FIG. 6 shows a further embodiment of the female Luer Lock connector 12 for the injection device of this invention as shown in FIG. 1. The connector 12 of FIG. 6 is distinguished by additional wings 128a, 128b, which are formed in one place onto the insertion region 121.

In FIG. 7, the use of the injection device 1 of this invention is shown schematically. The injection device 1 of this invention, with the hollow needle 20, needle holder 10, connecting tube 11 and connector 12, forms one part, in which the connecting tube 11 is joined solidly and in particular nondetachably both to the connector 12 and to the needle holder 10. The syringe 40, with a barrel 42 and plunger 41, can be prefilled with a powdered medication and is filled immediately before use with sterile water, so that the finished syringe 40 filled with injection solution 50 is now available. The syringe 40 is now introduced with its tip 43 directly into the female Luer Lock connector 12 into the widened bore 125a, and the injection solution can be injected through the connector 12, the connecting tube 11 and the hollow needle 20 into the patient by pressing on the plunger 41 in the direction of the arrow P2. The injection device 1 can be fixed to the surface of the patient's skin near the injection site with a bandage 60. The filter element 13 in the connector 12 reliably traps solid particles that get into the connector 12 along with the injection solution, so that even when the syringe 40 is filled with injection solution or sterile water, no unwanted solid particles can pass along with the injection solution into the circulatory system.

This invention enables simple handling, even by the patient and in emergency situations, of a syringe particularly for injecting powdered medications that must first be dissolved in sterile water. With the injection device of this invention with the winged cannula, connecting tube and connector with the built-in filter element, solid particles are removed with certainty from the injection solution.

German Patent Reference 202 07 083.2 and German Patent Reference 202 08 105.2, the priority documents corresponding to this invention, and their teachings are incorporated, by reference, into this specification.

1. In an injection device for injecting an injection solution prepared from a powdered medication in sterile water immediately before use which is administered by a syringe, and having a winged cannula (100) with a hollow needle (20) and a needle holder (10) with wings and including a female Luer Lock connector (12) joined to the winged cannula (100) by a flexible connecting tube (11) and having an insertion region (121) for the connecting tube (11) and a connection region (120) for placing the syringe (40), filled with the injection solution (50), against a skin surface, and having a continuous bore (125) that is continuous from the insertion region (121) to the connection region (120) of the Luer Lock connector (12), the improvement comprising: a porous filter element (13) permeable to the injection solution (50) provided in the continuous bore (125) of the Luer Lock connector (12).

2. In the injection device of claim 1, wherein the filter element (13) has pores with a mean diameter of 2 to 100 μm.

3. In the injection device of claim 2, wherein the filter element (13) has pores with a mean diameter of 5 to 20 μm.

4. In the injection device of claim 3, wherein the filter element (13) is disposed at a transition from the insertion region (121) to the connection region (120) of the Luer Lock connector (12), and the continuous bore (125) widens at the transition to the connection region (120) and forms a steplike shoulder (126).

5. In the injection device of claim 4, wherein the filter element (13) is embedded along an outer circumference of the filter element (13) in the Luer Lock connector (12).

6. In the injection device of claim 5, wherein the filter element (13) includes a woven fabric.

7. In the injection device of claim 5, wherein the filter element (13) has a disk of one of a sintered plastic granulate and a metal powder.

8. In the injection device of claim 7, wherein the connecting tube (11) is of a flexible plastic and has a length of 30 to 400 mm.

9. In the injection device of claim 8, wherein the connecting tube (11) has an outer diameter of 1.0 to 2.5 mm and an inner diameter of 0.2 to 1.5 mm.

10. In the injection device of claim 9, wherein the Luer Lock connector (12) on an outside of the insertion region (121) has two formed-on wings (128a, 128b) located in a same plane.

11. In the injection device of claim 10, wherein the Luer Lock connector (12) is of a thermoplastic, including one of an acrylate-butadiene-styrene, a polycarbonate, and a polymethacrylate.

12. In the injection device of claim 11, wherein the female Luer Lock connector (12) with the filter element (13) is formed of a first thermoplastic injection-molded part forming the insertion region (121) integrated with the filter element (13) placed on the inlet side (126a) of the insertion region (121) by spraying on a second injection-molded part comprising a thermoplastic, and the second injection-molded part forms the connection region (120).

13. In the injection device of claim 12, wherein an active region of the hollow needle (20) of the winged cannula (100) has a length (A) of 15 to 30 mm.

14. In the injection device of claim 13, wherein the hollow needle (20) has a removable protective sheath (30).

15. In the injection device of claim 14, wherein the needle holder (10) of the winged cannula (100) has a protective cover for covering the tip of the hollow needle (20) after use.

16. In the injection device of claim 1, wherein the filter element (13) has pores with a mean diameter of 5 to 20 μm.

17. In the injection device of claim 1, wherein the filter element (13) is disposed at a transition from the insertion region (121) to the connection region (120) of the Luer Lock connector (12), and the continuous bore (125) widens at the transition to the connection region (120) and forms a steplike shoulder (126).

18. In the injection device of claim 1, wherein the filter element (13) is embedded along an outer circumference of the filter element (13) in the Luer Lock connector (12).

19. In the injection device of claim 1, wherein the filter element (13) includes a woven fabric.

20. In the injection device of claim 1, wherein the filter element (13) has a disk of one of a sintered plastic granulate and a metal powder.

21. In the injection device of claim 1, wherein the connecting tube (11) is of a flexible plastic and has a length of 30 to 400 mm.

22. In the injection device of claim 1, wherein the connecting tube (11) has an outer diameter of 1.0 to 2.5 mm and an inner diameter of 0.2 to 1.5 mm.

23. In the injection device of claim 1, wherein the Luer Lock connector (12) on an outside of the insertion region (121) has two formed-on wings (128a, 128b) located in a same plane.

24. In the injection device of claim 1, wherein the Luer Lock connector (12) is of a thermoplastic, including one of an acrylate-butadiene-styrene, a polycarbonate, and a polymethacrylate.

25. In the injection device of claim 1, wherein the female Luer Lock connector (12) with the filter element (13) is formed of a first thermoplastic injection-molded part forming the insertion region (121) integrated with the filter element (13) placed on an inlet side (126a) of the insertion region (121) by spraying on a second injection-molded part comprising a thermoplastic, and the second injection-molded part forms the connection region (120).

26. In the injection device of claim 1, wherein an active region of the hollow needle (20) of the winged cannula (100) has a length (A) of 15 to 30 mm.

27. In the injection device of claim 1, wherein the hollow needle (20) has a removable protective sheath (30).

28. In the injection device of claim 1, wherein the needle holder (10) of the winged cannula (100) has a protective cover for covering the tip of the hollow needle (20) after use.
